(19) United States
(12) Patent Application Publication
(10) Pub. No.: US 2024/0349832 A1
(43) Pub. Date: Oct. 24, 2024

(54) SYSTEMS AND APPARATUSES FOR STABILIZING A DIGIT

(71) Applicant: James Perez, Fresno, CA (US)

(72) Inventor: James Perez, Fresno, CA (US)

(21) Appl. No.: 18/640,284

(22) Filed: Apr. 19, 2024

Related U.S. Application Data
(60) Provisional application No. 63/497,097, filed on Apr. 19, 2023.

(51) Int. Cl. A41D 19/015

(52) U.S. Cl. CPC A41D 19/01582

(57) ABSTRACT

An apparatus for use with an athletic glove which helps stabilize a digit, in particular, a thumb, as a hand is moved in a variety of motions, such as swinging a golf club. The apparatus has a sleeve at a first end in which a portion of the digit may be placed. The body of the apparatus is positioned across the palm side of the hand, around a lateral side thereof, and around the backside thereof. A second end of the apparatus may be inserted through an opening in an athletic glove, between the wrist strap and wrist flap thereof. The second end may have a fastener configured to engage fasteners of the wrist strap and wrist flap of the glove. A stabilizing insert may be engaged with the body of the apparatus and partially inserted into the sleeve thereof, before being placed around a thumb.

### CROSS-REFERENCE TO RELATED APPLICATIONS

This application claims priority to, and the benefit of, U.S. Provisional Patent Application No. 63/497,097, filed Apr. 19, 2023, which is hereby incorporated by reference in its entirety.

### FIELD OF THE INVENTION

The present invention concerns an apparatus for supporting and stabilizing a digit. More particularly, some embodiments of the present invention concern an apparatus having a sleeve at one end which may be placed over a digit and, at another end, secured to an athletic glove. The apparatus, in conjunction with an athletic glove, may provide stability and support to a digit during a range of motions.

### BACKGROUND OF THE INVENTION

In sports such as golf and baseball, repetitive motions and impacts can cause pain and injury to a person's hand and/or digits. With respect to golf, the thumb of a player's non-dominant hand (e.g., the left hand for a right-handed golfer) plays an important role in the golf swing, as it helps supports and stabilize the club shaft along the grip. Therefore, the thumb requires both strength and stability in order to make a proper swing.

As a result of the repetitive motion and strain on the thumb, as well as repeated impact with the ground, thumb area injuries in golf are common. The risk of such injury increases with the use of training mats (i.e., artificial turf), which generally increases the force of impact on the club head, relative to natural turf. Poor swing technique can also raise injury risk, thereby affecting amateur golfers, or those who might not be physically able to make a proper swing (such as older populations). Over time, damage can occur to tendons and ligaments in the hand which can hinder the ability to swing a golf a club, or force a player to stop altogether.

One conventional means for relieving pain, or to avoid injury, involves the use of a splint around the thumb to stabilize and inhibit motion of the thumb. However, a splint is impractical for use during a round of golf or practice, as a player is unable to wear a glove and splint at the same time. Even without a glove, a splint is too restrictive on the thumb, making it difficult to near impossible to properly swing a club. Another conventional method is to wrap athletic tape around the thumb and adjacent area to help stabilize, and restrict movement of, the thumb. However, athletic tape can be cumbersome to apply and remove, as it is needs to be wrapped in a certain configuration and to a certain tightness to be effective. As with the conventional approach of using a split, simultaneous use of athletic tape and a glove is difficult. Furthermore, athletic tape has limited usability, requiring periodic replacement or adjustment, which can be difficult during a round of golf, for example.

Therefore, the need exists for a device which can support and stabilize a thumb during a range of motions, such as swinging a golf club, baseball bat, tennis racket, or the like. Furthermore, the need exists for a device which can be used with existing athletic gloves, is reusable, and is easy to wear and remove.

### BRIEF SUMMARY OF THE INVENTION

The present invention generally comprises an apparatus, hereinafter referred to as a “brace”, for stabilizing a digit. The present invention also comprises a system for stabilizing a digit which comprises a brace used in combination with an athletic glove. According to some implementations, a brace may be worn over a digit (in preferred implementations, a thumb), wrapped around a side of a hand, and attached, or otherwise engaged, to one or more fasteners of a glove being worn over the hand.

In accordance with some embodiments of the present invention, a brace may be generally elongated, flexible, and thin (i.e., having two substantially parallel, planar surfaces). According to some embodiments, the brace may have a first end configured to be engaged with a digit, such as a thumb, of a user and a second end configured to be engaged with a glove. In some embodiments, the second end may comprise a fastener which may be, preferably, configured to engage a corresponding fastener of a glove. More particularly, and according to some embodiments, the fastener of the brace may comprise a male (female) portion on a first side thereof configured to mate with a female (male) portion of the glove fastener. Similarly, in some embodiments, the fastener may comprise a female (male) portion on a second side of the brace configured to mate with a male (female) portion of the glove fastener.

In accordance with some embodiments of the present invention, the first end of the brace may comprise a sleeve having an opening within which a digit may be received and retained. In some embodiments, the opening of the sleeve may be disposed on the first side of the brace such that the sleeve, as viewed from the second side of the brace, is continuous with the second side of the brace. According to some embodiments, the sleeve may be engaged with a digit and a body of the brace—which may be generally defined as the section of the brace between the first and second ends—may rest across a portion a user's hand.

According to some embodiments of the present invention, the brace may be used in combination with a stabilizing insert. In some embodiments, the insert may be generally thin and flexible, having a shape that is generally the same as a body of the brace. In preferred embodiments, the insert may be less pliable relative to the brace, so as to provide more rigidity to the brace (and thus more stability). In some embodiments, the insert may have an adhesive backing configured to adhere to a portion of the brace. According to some embodiments, the insert may be placed on, and, in some embodiments, adhered to, a portion of the first side of the brace, with an end of the insert disposed within the sleeve of the brace. In some embodiments, the insert may be slightly curved such that a first side may be concavely rounded and a second side may be convexly rounded.

In accordance with some embodiments of the present invention, the brace may be used in combination with a glove. In preferred embodiments, a glove may be an athletic glove having at least one sheath for receiving a digit, a strap, and one or more fasteners for securing the strap. In some embodiments, an opening may be formed generally at a wrist portion of the glove and adjacent to a base of the strap. The brace may be adapted such that a portion thereof may be disposed within the opening.

In preferred implementations of the present invention, a user may use a brace in combination with a glove to stabilize a digit, preferably a thumb. For example, when worn, the sleeve of the brace may be around the user's thumb and a portion of the body of the brace may rest along, or abut, a user's palm, wrapping around a lateral side of the user's hand. When worn over the user's hand, the glove may envelop the sleeve of the brace and most of the remaining portion of the brace. The second end of the brace may be disposed within the opening near the wrist portion of the glove. The fastener of the brace may be attached to a pair of fastening elements of the glove, where the brace fastener is positioned and secured between a first fastening element on a strap of the glove and a second fastening element on the wrist portion of the glove. By means of the attachment of the fastening end of the brace to the glove and the securing of the sleeve end around the user's digit, any motion of the digit which may be generally in a direction away from the lateral side of the user's hand may be opposed, at least partially, by the force of the brace along the lateral side of the user's hand.

According to some implementations, a method for stabilizing a digit using the brace may include, first, positioning a portion of the digit within the sleeve of the brace and orienting the brace such that the body thereof is on the palm side of the user's hand. Next, the brace may be wrapped around the lateral side of the user's hand, opposite to the user's thumb. Next, the user may place a glove around the user's hand such that a sheath of the glove envelopes the user's thumb. Next, the user may guide the fastening end of the brace through the opening between the strap and wrist portion of the glove. Next, in one implementation, a user may pull the brace until there is no slack in the brace. Next, the user may engage the fastening end of the brace to the glove by attaching the first fastening element of the brace to the strap fastening element of the glove and attaching the second fastening element of the brace to the wrist fastening element of the glove (or vice versa).

In accordance with some embodiments of the present invention, a brace for stabilizing a digit may comprise: a sleeve disposed at a first end which may comprise an opening therein which may be configured to receive a portion of the digit a body generally extending from the first end to a second end; and a fastener at the second end, wherein the fastener may comprise at least one fastening element; wherein the opening of the sleeve may be disposed at a first side of the brace. In some embodiments, the opening of the sleeve may be configured to receive a distal end of a thumb. In some embodiments, the fastener may be configured to engage at least one fastener of an athletic glove. In some embodiments, the fastener may consist of one of the group consisting of hooks and loops. In some embodiments, the brace may further comprise a stabilizing insert engaged with the body. In some embodiments, the stabilizing insert may comprise neoprene. In some embodiments, the stabilizing insert may comprise an adhesive for adhering to the body. In some embodiments, each of the sleeve and the body may be flexible. In some embodiments, each of the sleeve and the body may comprise fabric.

In accordance with some embodiments of the present invention, a system for stabilizing a digit of a hand may comprise: a) an athletic glove which may comprise: a plurality of sheaths; a wrist strap which may comprise a fastener; a wrist flap which may comprise a fastener, wherein the fastener of the wrist strap may be removably engaged to the fastener of the wrist flap; and an opening between the wrist strap and the wrist flap; b) a brace which may comprise: a first side and a second side; a first end and a second end; a sleeve which may be disposed at the first end and may comprise an opening therein, where the opening may be on the first side; a flexible body extending from the first end to the second end; and a first fastener on the first side of the brace and a second fastener on the second side of the brace; wherein the second end of the brace may be configured to be received through the opening of the glove, wherein the first fastener of the brace may be removably engaged with the fastener of the wrist strap, wherein the second fastener of the brace may be removably engaged with the fastener of the wrist flap, and wherein the sleeve may be configured to be received within a first one of the sheaths. In some embodiments, the glove may comprise a golfers glove. In some embodiments, the sleeve of the brace and the first sheath of the glove may be each configured to receive a thumb. In some embodiments, the system may further comprise a stabilizing insert engaged with the brace. In some embodiments, the stabilizing insert may comprise an adhesive for adhere to the first side of the brace. In some embodiments, the stabilizing insert may comprise neoprene.

In accordance with some embodiments of the present invention, a method for stabilizing a thumb of a hand using a brace and an athletic glove, may comprise the steps of: a) positioning a sleeve of the brace over a distal portion of the thumb, wherein the sleeve may be disposed at a first end of the brace and wherein an opening of the sleeve may be positioned at a back side of the thumb; b) positioning a first portion of the brace across a palm side of the hand; c) positioning a body of the brace around a lateral side of the hand; d) positioning the body of the brace across a back side of the hand; e) positioning the hand in the athletic glove, and positioning the thumb and the sleeve within a sheath of the glove; f) positioning a second end of the brace through an opening between a wrist strap and a wrist flap of the glove; and g) engaging at least one fastener of the brace with a fastener of one of the group consisting of the glove wrist strap, the glove wrist flap, and combinations thereof. In some embodiments, the step of engaging the at least one fastener of the brace with the fastener of one of the group consisting of the glove wrist strap, the glove wrist flap, and combinations thereof, may comprise engaging a first fastening element of the at least one fastener of the brace with the fastener of the wrist flap and engaging the fastener of the wrist strap with a second fastening element of the at least one fastener of the brace. In some embodiments, the method may further comprise the step of removing slack from the brace, wherein the step of removing slack from said brace may be performed before the step of engaging the at least one fastener of the brace with the fastener of one of the group consisting of the glove wrist strap, the glove wrist flap, and combinations thereof. In some embodiments, the method may further comprise the step of engaging a stabilizing insert with the brace on a first side thereof.

### BRIEF DESCRIPTION OF THE DRAWINGS

FIG. 1 is a first side view diagram illustrating an exemplary brace for stabilizing a digit, in accordance with some embodiments of the present invention.

FIG. 2 is a second side view diagram illustrating the brace of FIG. 1, in accordance with some embodiments of the present invention.

FIG. 3 is a diagram illustrating a portion of the brace of FIG. 1 engaged with a left hand, as viewed from a palm side of the hand, in accordance with some embodiments of the present invention.

FIG. 4 is a diagram illustrating a portion of the brace of FIG. 1 engaged with the hand of FIG. 3, as viewed from a back side of the hand, in accordance with some embodiments of the present invention.

FIG. 5 is a diagram illustrating a portion of the brace of FIG. 1 and an exemplary glove worn over a hand, as viewed from a back side of the glove, in accordance with some embodiments of the present invention.

FIG. 6 is a diagram illustrating a portion of the brace of FIG. 1 engaged with the glove of FIG. 5 glove, as viewed from the back side of the glove, in accordance with some embodiments of the present invention.

FIGS. 7A and 7B are diagrams illustrating a first side and a second side, respectively, of an exemplary stabilizing insert, in accordance with some embodiments of the present invention.

FIG. 8 is a diagram illustrating the stabilizing insert of FIGS. 7A and 7B with an exemplary brace, in accordance with some embodiments of the present invention.

### DETAILED DESCRIPTION OF THE INVENTION

The invention, in its various aspects, will be explained in greater detail below. While the invention will be described in conjunction with several exemplary embodiments, the exemplary embodiments themselves do not limit the scope of the invention. Similarly, the exemplary illustrations in the accompanying drawings, where like elements have like numerals, do not limit the scope of the exemplary embodiments and/or invention, including any length, angles, or other measurements provided. Rather the invention, as defined by the claims, may cover alternatives, modifications, and/or equivalents of the exemplary embodiments.

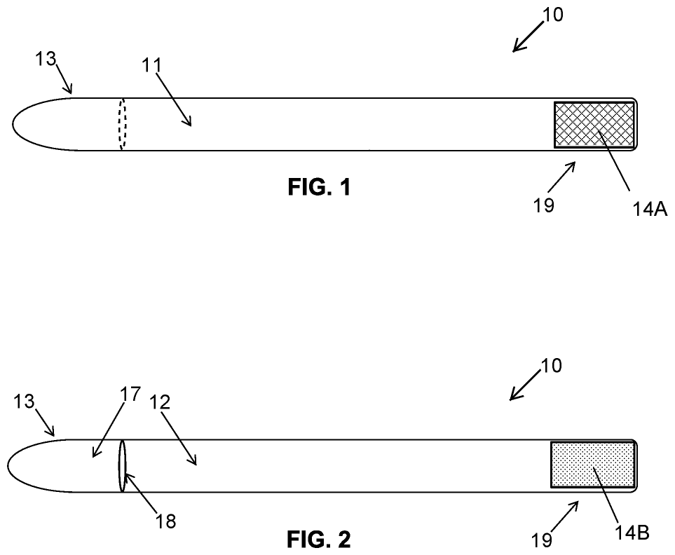

Referring, generally, to FIGS. 1 and 2, and in accordance with some embodiments of the present invention, a brace 10 may be generally elongated and thin, and having a first side 11 and a second side 12, where a surface of first side 11 may be about parallel to a surface of second side 12. In some embodiments, brace 10 may comprise a first end 13 which may comprise a sleeve 17 which may be configured to be engaged with a digit of a hand. Brace 10 may also, according to some embodiments, have a second end 19 which may comprise a fastener consisting of fastening elements 14A and 14B (or, in some embodiments, a single fastening element) which may be configured to be engaged with fastener of a glove. In some embodiments, a fastener may comprise a hook-and-loop type fastener, buttons, snaps, or the like. For example, and without limitation, a fastening element of a brace may comprise a plurality of hooks configured to engage a plurality of loops of a fastener of a glove.

As illustrated more clearly in FIG. 2, for example, sleeve 17 may have an opening 18 on second side 12 of brace 10 through which a distal portion of a digit of a hand may be received and retained therein. According to some embodiments, since opening 18 of sleeve 17 may be on second side 12, the portion of sleeve 17 which is on first side 11 of brace 10 may be continuous with the surface of first side 11, as illustrated, for example, in FIG. 1 (where broken lines illustrate the position of opening 18 on the opposite side of brace 10, relative to FIG. 2).

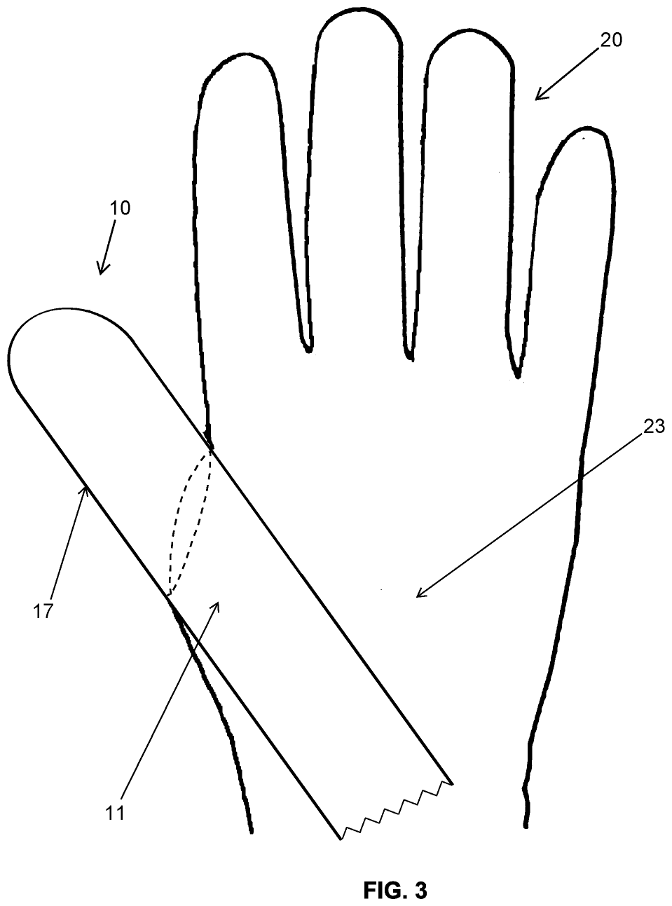

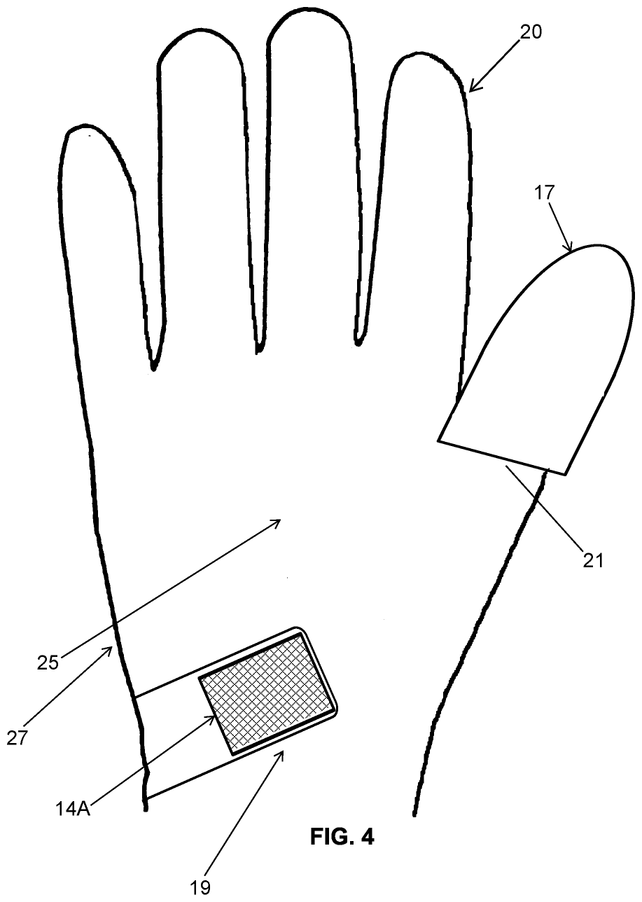

Referring now, generally, to FIGS. 3 and 4, for example, and in accordance with some embodiments, brace 10 may be engaged with a thumb 21 of a hand 20. For example, sleeve 17 may be placed around a distal end of thumb 21 such that the distal end of thumb 21 is received through opening 18 of brace 10 (illustrated in broken lines in FIG. 3). According to some embodiments, brace 10 may be preferably positioned such that a portion of brace 10 extends across a palm side 23 of hand 20, with the surface of first side 11 facing away from palm side 23 of hand 20, as illustrated in FIG. 3. In some embodiments, and as illustrated FIG. 4, for example, as viewed from a back side 25 of hand 20, first end 13 of brace 10 may be secured to thumb 21 by sleeve 17 with a portion of brace 10 wrapping around a lateral side 27 (i.e., the side opposite to thumb 21). According to some embodiments, when brace 10 is wrapped around lateral side 27, second end 19 may rest along a surface of back side 25, near the wrist of hand 20, with fastening element 14A facing away from the surface of back side 25.

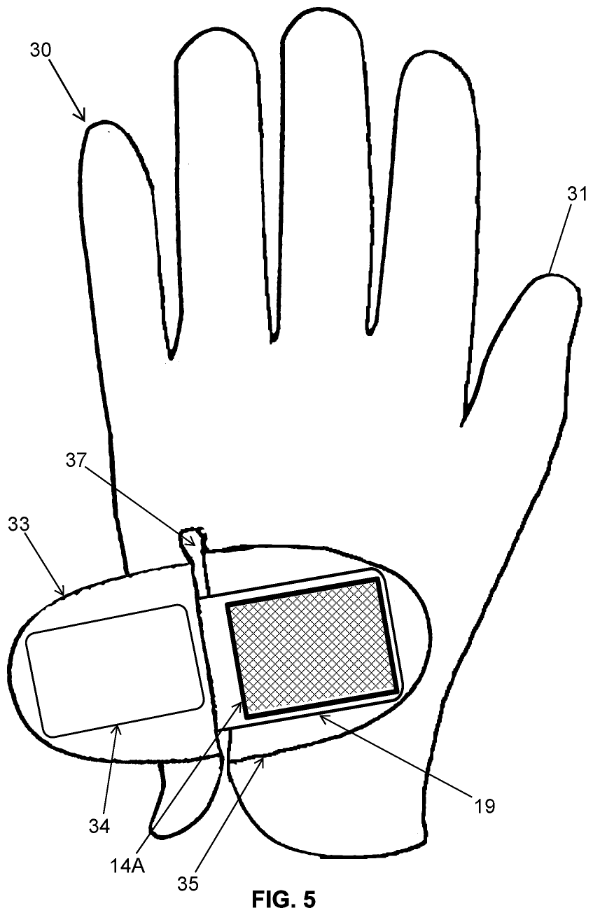

According to preferred implementations, and with reference, for example, to FIG. 5, brace 10 may be used in combination with an athletic glove 30 which may be, but is not limited to, a golf glove or batting glove. Glove 30 may comprise a plurality of sheaths 31 covering the digits of a hand (not illustrated) and a wrist strap 33 having a fastener 34 which may be removably engaged with a fastener of a wrist flap 35 of glove 30 (the fastener being underneath second end 19 of brace 10 in FIG. 5) in order to secure wrist strap 33 to wrist flap 35. As further illustrated in FIG. 5, and in accordance with some embodiments, an opening 37 may exist between wrist flap 35 and a base of wrist strap 33 which may extend, generally, along and near a portion of a lateral side of glove 30. In some embodiments, opening 37 may be adapted such that second end 19 of brace 10 may be guided therethrough and a portion of brace 10 may be disposed therein, as further illustrated in FIG. 5.

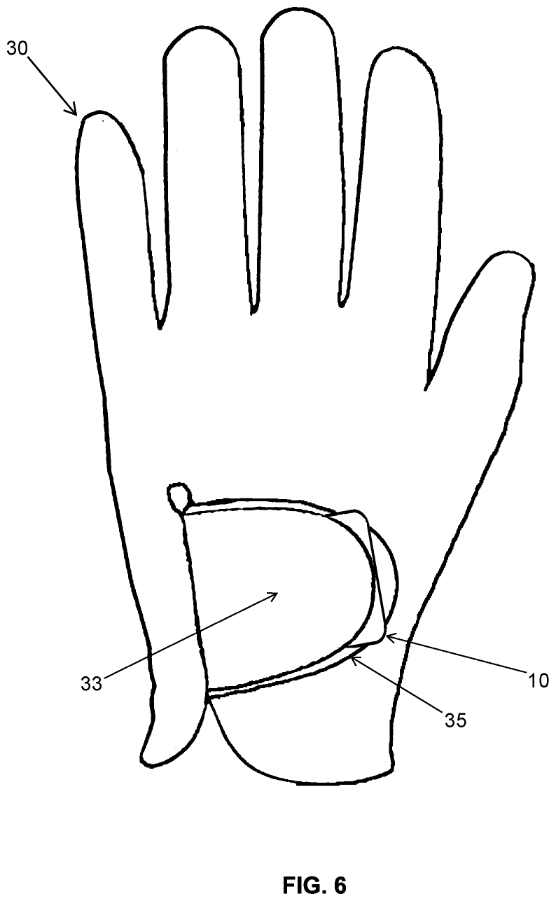

In accordance with preferred implementations, and as further illustrated in FIG. 5, when glove 30 is worn over a hand, sleeve 17 (not illustrated) of brace 10 may be enveloped by sheath 31 and a majority of the remaining portion of brace 10 may be enveloped by a palm side of glove 30. With reference to FIGS. 5 and 6, and according to some embodiments, brace 10 may be secured to glove 30 by the engagement of both i) fastening element 14A of brace 10 with fastener 34 of wrist strap 33 and ii) fastening element 14B (illustrated in FIG. 2) with the fastener of wrist flap 35 (not illustrated; disposed behind brace 10), such that second end 19 of brace 10 may be disposed between wrist strap 33 and wrist flap 35. It is to be appreciated that, and according to some embodiments, a fastener of a brace may comprise one fastening element which may engage a fastener of a wrist strap or a fastener of a wrist flap. For example, a fastening element may be configured to engage a fastener of a wrist strap (or wrist flap), but may be smaller (i.e., narrower) than the fastener of the wrist strap. In this case, the fastener of the wrist strap may be able to engage both the fastening element of the brace and the fastener of the wrist flap (assuming the fastener of the wrist flap is at least bigger (i.e., wider) than the fastening element. Although partially shown in the illustration of FIG. 6, it is to be appreciated that, according to some implementations, a brace may not be visible when disposed between a wrist strap and a wrist flap when viewed from a back side of a glove (e.g., the view illustrated in FIG. 6).

It is to be appreciated that, and with further reference to FIG. 6, by anchoring second end 19 of brace 10 between wrist strap 33 and wrist flap 35, the range of motion of a thumb (not illustrated) may be restricted or reduced. For instance, if the thumb is extended or abducted away from the palm, this motion may be restricted by opposing forces along the lateral side of the hand and at the anchor point of brace 10 to glove 30. This may be advantageous in that it may prevent hyperextension of the thumb (and thus avoid potential injury thereto).

Further to the above, when gripping an object, such as a club or a bat, the forces acting on a thumb by virtue of the brace may cause the thumb to apply pressure against the object (or vice versa), thus reducing the ability for the thumb to extend or abduct. This may be important when a user moves the object with a substantial amount of speed and torque as the brace may stabilize the thumb during the motion and prevent potential injury that could arise from the force that the object exerts on the user's thumb—either due to an isolated incident (e.g., hyperextension) or due to repetitive motion. Furthermore, the brace may stabilize the thumb during impact of the object with another object, such as during impact of a bat with a baseball, or during impact of a golf club with the ground.

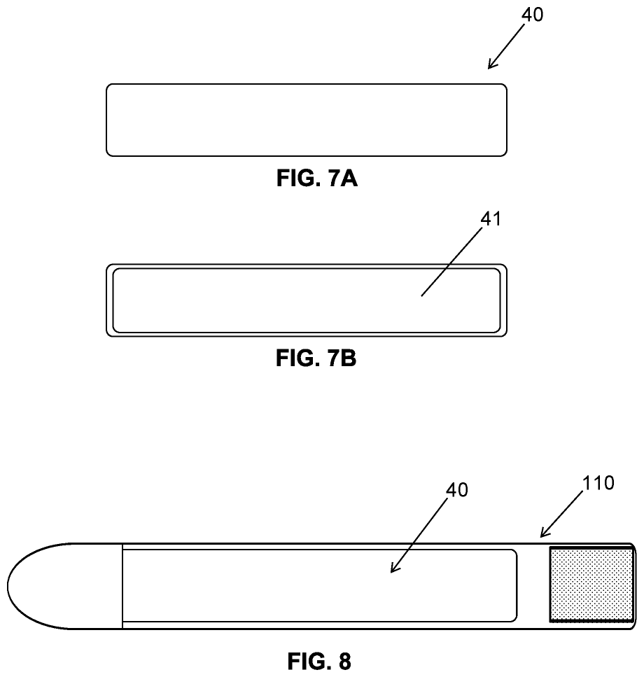

In accordance with some embodiments of the present invention, a brace may be used in combination with a stabilizing insert. For example, and as illustrated in FIGS. 7A and 7B, a stabilizing insert 40 may be generally thin and flexible, having a shape similar to a body of a brace. According to some embodiments, a stabilizing insert may be constructed with material that may be relatively more rigid, durable, and tacky than that of the brace material. For example, a stabilizing insert may be constructed with natural and/or synthetic rubber, such as neoprene, or the like. In some embodiments, a stabilizing insert may be configured to adhere to a portion of a brace. For example, and with further reference to FIG. 7B, insert 40 may comprise an adhesive backing 41 which may allow insert 40 to be adhered to a side of a brace. In some embodiments, one end of an insert may disposed within a sleeve of a brace. For example, and with reference to FIG. 8, insert 40 may be adhered to a brace 110 on the side thereof which comprises the sleeve opening. In some embodiments, an insert may be slightly curved such that a side may be concavely rounded and a second side may be convexly rounded, which may help the insert to contour the shape of a digit.

According to some implementations, and with reference, generally, to FIGS. 1-6, to use brace 10 to stabilize thumb 21, a user may position thumb 21 within sleeve 17 by inserting thumb 21 through opening 18 and orienting brace 10 (if not already properly oriented) such that the surface of first side 11 is away from palm side 23 of hand 20 with a portion of the second side 12 against the palm of hand 20 (see, e.g., FIG. 3). Brace 10 may be wrapped loosely around lateral side 27 of hand 20 such that, if second end 19 is laid across back side 25 of hand 20, fastening element 14A may be exposed (see, e.g., FIG. 4). The user may place glove 30 around hand 20 such that sheath 31 is around thumb 21 (and, thus, sleeve 17), without engaging wrist strap 33 to wrist flap 35. The user may guide second end 19 of brace 10 through opening 37 of glove 30 (see, e.g., FIG. 5). The user may engage both i) fastening element 14B (not illustrated) of brace 10 to the fastener of wrist flap 35 (pulling on first end 13, if needed, in order to reduce or remove slack prior to engaging the fasteners) and ii) fastening element 14A of brace 10 to fastener 34 of strap 33 of glove 30 (or vice versa).

It is to be understood that variations, modifications, and permutations of embodiments of the present invention may be made without departing from the scope thereof. It is also to be understood that the present invention is not limited by the specific embodiments, descriptions, or illustrations or combinations of either components or steps disclosed herein. Thus, although reference has been made to the accompanying figures, it is to be appreciated that these figures are exemplary and are not meant to limit the scope of the invention. In particular, it is to be appreciated that a brace, as described heretofore, may be adapted for various sports, such as, but not limited to golf, baseball, lacrosse, or tennis. However, it is also to be appreciated that the brace can be used in any situation where a user desires stabilization of a digit. For example, a person with a minor injury to a thumb may use the brace to stabilize the thumb and restrict its motion as they go about daily activities. As another example, a person with trigger finger may use the brace to alleviate symptoms. It is also to be appreciated that a brace may be adapted or modified to be engaged with articles other than gloves. For example, a brace may be adapted or modified to be engaged with a wrist strap or band, or the like, having a fastener.

What is claimed is:

1. A brace for stabilizing a digit, comprising:
a) a sleeve disposed at a first end and comprising an opening therein configured to receive a portion of said digit;
b) a body extending from said first end to a second end; and
c) a fastener at said second end, said fastener comprising at least one fastening element;
wherein said opening of said sleeve is disposed at a first side of said brace.

2. The brace of claim 1, wherein said opening of said sleeve is configured to receive a distal end of a thumb.

3. The brace of claim 1, wherein said fastener is configured to engage at least one fastener of an athletic glove.

4. The brace of claim 1, wherein said fastener consists of one of the group consisting of hooks and loops.

5. The brace of claim 1, further comprising a stabilizing insert engaged with said body.

6. The brace of claim 5, wherein said stabilizing insert comprises neoprene.

7. The brace of claim 5, wherein said stabilizing insert comprises an adhesive for adhering to said body.

8. The brace of claim 1, wherein each of said sleeve and said body are flexible.

9. The brace of claim 8, wherein each of said sleeve and said body comprise fabric.

10. A system for stabilizing a digit of a hand, comprising:
a) an athletic glove comprising:
i) a plurality of sheaths;
ii) a wrist strap comprising a fastener;
iii) a wrist flap comprising a fastener, wherein said fastener of said wrist strap is removably engaged to said fastener of said wrist flap; and
iv) an opening between said wrist strap and said wrist flap;
b) a brace comprising:
i) a first side and a second side;
ii) a first end and a second end;
iii) a sleeve disposed at said first end and comprising an opening therein, said opening on said first side;
iv) a flexible body extending from said first end to said second end; and
v) a first fastener on said first side of said brace and a second fastener on said second side of said brace;
wherein said second end of said brace is configured to be received through said opening of said glove,
wherein said first fastener of said brace is removably engaged with said fastener of said wrist strap,
wherein said second fastener of said brace is removably engaged with said fastener of said wrist flap, and
wherein said sleeve is configured to be received within a first one of said sheaths.

11. The system of claim 10, wherein said glove comprises a golfers glove.

12. The system of claim 10, wherein said sleeve of said brace and said first sheath of said glove are each configured to receive a thumb.

13. The system of claim 10, further comprising a stabilizing insert engaged with said brace.

14. The system of claim 13, wherein said stabilizing insert comprises an adhesive for adhere to said first side of said brace.

15. The system of claim 13, wherein said stabilizing insert comprises neoprene.

16. A method for stabilizing a thumb of a hand using a brace and an athletic glove, comprising the steps of:
a) positioning a sleeve of said brace over a distal portion of said thumb, wherein said sleeve is disposed at a first end of said brace and wherein an opening of said sleeve is positioned at a back side of said thumb;
b) positioning a first portion of said brace across a palm side of said hand;
c) positioning a body of said brace around a lateral side of said hand;
d) positioning said body of said brace across a back side of said hand;
e) positioning said hand in said athletic glove, and positioning said thumb and said sleeve within a sheath of said glove;
f) positioning a second end of said brace through an opening between a wrist strap and a wrist flap of said glove; and
g) engaging at least one fastener of said brace with a fastener of one of the group consisting of said glove wrist strap, said glove wrist flap, and combinations thereof.

17. The method of claim 16, wherein said step of engaging said at least one fastener of said brace with said fastener of one of the group consisting of said glove wrist strap, said glove wrist flap, and combinations thereof, comprises engaging a first fastening element of said at least one fastener of said brace with said fastener of said wrist flap and engaging said fastener of said wrist strap with a second fastening element of said at least one fastener of said brace.

18. The method of claim 16, further comprising the step of removing slack from said brace, wherein said step of removing slack from said brace is performed before said step of engaging said at least one fastener of said brace with said fastener of one of the group consisting of said glove wrist strap, said glove wrist flap, and combinations thereof.

19. The method of claim 16, further comprising the step of engaging a stabilizing insert with said brace on a first side thereof.
